(19) United States
(12) Patent Application Publication
(10) Pub. No.: US 2011/0287503 A1
(43) Pub. Date: Nov. 24, 2011

(54) METHODS FOR PRODUCING HYDROCARBON PRODUCTS FROM ALGAL BIOMASS

(75) Inventors: Francis S. Lupton, Evanston, IL (US); Thomas J. Traynor, Vernon Hills, IL (US)

(73) Assignee: UOP LLC, Des Plaines, IL (US)

(21) Appl. No.: 12/784,336

(22) Filed: May 20, 2010

(51) Int. Cl. C12P 5/02; C12P 5/00

(52) U.S. Cl. 435/167; 435/166

(57) ABSTRACT

Methods for producing hydrocarbon oils from algal biomass are provided. The algal biomass is hydrogenolysed under reaction conditions sufficient to produce a partially deoxygenated lipid-based oil. The algal biomass may be whole algal biomass, residual algal biomass, or both. The algal biomass is hydrogenolysed by liquefying the algal biomass in the presence of a hydrogenolytic catalyst in a hydrogen atmosphere at an elevated temperature and pressure to produce an organic phase containing the partially deoxygenated lipid-based oil, an aqueous phase, and a solid phase. The aqueous and solid phases may be removed from the partially deoxygenated lipid-based oil. The partially deoxygenated lipid-based oil is then substantially deoxygenated using a hydroprocessing catalyst to produce the hydrocarbon oil.

### FIELD OF THE INVENTION

The present invention generally relates to biofuels, and more particularly relates to methods for producing hydrocarbon products from algal biomass.

### DESCRIPTION OF RELATED ART

Alternative sources for petroleum fuel are being sought as natural oil supplies are being depleted, petroleum costs are increasing, concerns about pollution, and political pressure to decrease dependence on foreign fuel stock. Biofuels are derived from biomass and are intended to provide an alternative to petroleum fuels. Biofuels can be burned directly as fuel for certain boiler and furnace applications, and can also serve as a potential feedstock in processes for the production of transportation fuels in petroleum refineries. Biomass can also be used to make other useful organic chemical products. Algae are one type of biomass that is of particular interest because they are one of the fastest growing plants on the planet therefore offering one of the highest yields per unit area. Algae also do not need arable land, and can be grown with impaired water.

Algae have been used as a feedstock to produce biofuel using various methods. Algae contain neutral lipids (triacelelydrides (TAGs)), glycolipids found in algal chloroplast membranes (e.g., monogalactosyldiacylglycerols and digalactosyldiacylglycerols), and polar lipids of the algal plasma membranes, primarily phospholipids (e.g., phosphatidylcholine). The glycolipids and other polar lipids represent a significant portion of the total lipids in the algae. The neutral lipids (hereinafter “TAG lipids”) in algae have been converted to FAME (fatty acid methyl esters) biodiesel using conventional lipid extraction with a solvent such as hexane and base-catalyzed transesterification methods.

FAME biodiesel has flow properties and ignition properties (cetane values) compatible with most diesel engines. This conventional production of biofuel from algae uses only a fraction (the neutral lipids) of the total available lipid material in the algae leaving a large percentage of “residual algal biomass” remaining after the TAG lipids have been extracted to form a neutral oil extract. This and other conventional methods are incapable of converting the glycolipids and other polar lipids in the algae into biofuel. These lipids cannot be extracted from algal biomass by conventional methods, and thus, cannot be transesterified. Therefore, conventional methods produce only a small fraction of the energy that can potentially be obtained from the algae.

The conventional methods also require the use of strong alkali catalysts which pose significant material handing and waste disposal problems and also do not directly produce a product that is a useable biofuel due to its high oxygen content. A separate deoxygenation process is typically required to convert the neutral oil extract obtained from conventional methods into a useable biofuel.

Accordingly, it is desirable to provide methods for producing biofuels from algae. It is also desired to convert more of the lipids available in the algae into a hydrocarbon product that can be processed into biofuels thus increasing the useful oil yield compared to conventional methods. It is also desired to produce a substantially deoxygenated hydrocarbon product in one step without the use of caustic chemicals. Furthermore, other desirable features and characteristics of the present invention will become apparent from the subsequent detailed description of the invention and the appended claims, taken in conjunction with the accompanying drawings and this background of the invention.

### SUMMARY OF THE INVENTION

Methods are provided for producing a hydrocarbon oil from algal biomass. In accordance with one exemplary embodiment, the method comprises hydrogenolysing the algal biomass under reaction conditions sufficient to produce a partially deoxygenated lipid-based oil. The partially deoxygenated lipid-based oil is substantially deoxygenated.

Methods are provided for producing a hydrocarbon oil from algal biomass, in accordance with yet another exemplary embodiment of the present invention. The method comprises liquefying the algal biomass in the presence of a hydrogenolytic catalyst and hydrogen gas to produce a partially deoxygenated lipid-based oil. The partially deoxygenated lipid-based oil is substantially deoxygenated using a hydroprocessing catalyst to produce the hydrocarbon oil.

Methods are provided for producing liquid hydrocarbons from algal biomass in accordance with yet another exemplary embodiment of the present invention. The method comprises liquefying whole algal biomass, residual algal biomass, or both, in the presence of a hydrogenolytic catalyst in a hydrogen atmosphere at an elevated temperature and pressure to produce an organic phase comprising a partially deoxygenated lipid-based oil, an aqueous phase, and a solid phase. The aqueous and solid phases are removed from the partially deoxygenated lipid-based oil. The partially deoxygenated lipid-based oil is substantially deoxygenated using a hydroprocessing catalyst.

### BRIEF DESCRIPTION OF THE DRAWINGS

The present invention will hereinafter be described in conjunction with the following drawing figures, wherein like numerals denote like elements, and wherein:

FIG. 1 is a flow chart of a method for producing a hydrocarbon oil, according to exemplary embodiments of the present invention;

FIG. 2 is a functional block diagram of an apparatus to convert algae to various biofuels, in accordance with exemplary embodiments of the present invention;

FIG. 3 is a table of suitable exemplary hydrogenolytic catalysts; and

FIGS. 4 and 5 are exemplary chemical diagrams illustrating the production of exemplary paraffin hydrocarbons from exemplary components of algal biomass.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Various exemplary embodiments of the present invention are directed to methods for producing biofuel from algal biomass. The algal biomass is hydrogenolysed in the presence of a hydrogenolytic catalyst to liquefy the algal biomass into a partially deoxygenated lipid-based oil and the partially deoxygenated lipid-based oil is substantially deoxygenated using a hydroprocessing catalyst to produce a hydrocarbon oil. The hydrocarbon oil can then be used as biofuels and/or processed by known methods into biofuels.

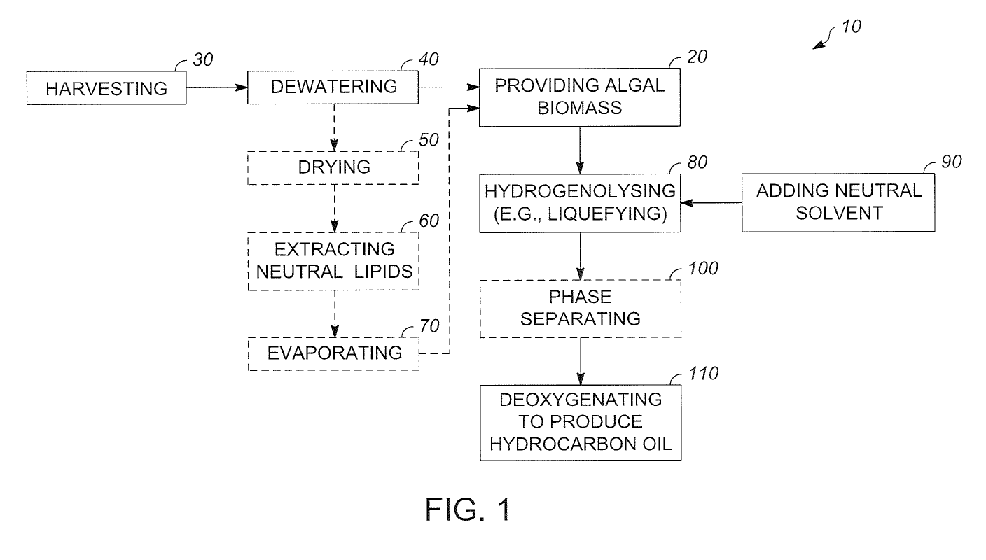

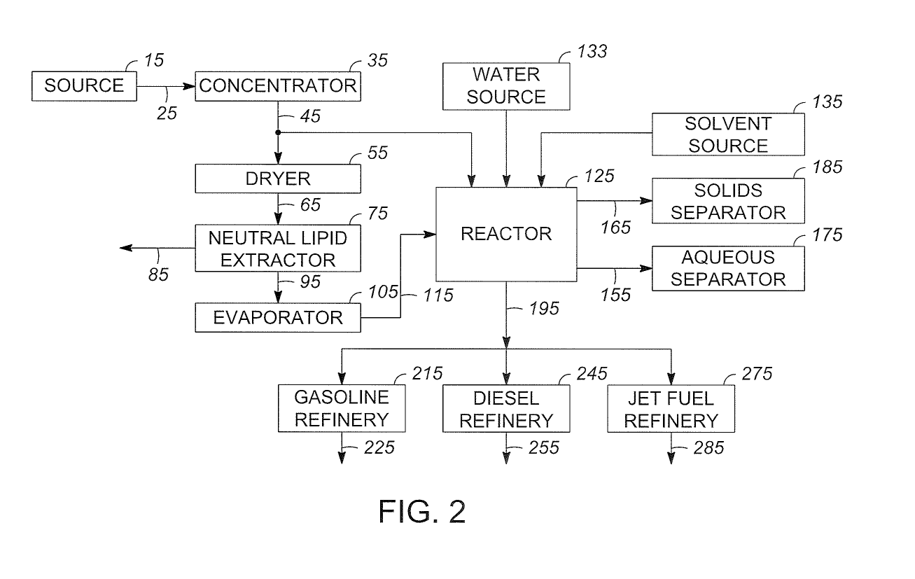

FIG. 1 is a flow chart of a method 10 for producing the hydrocarbon oil. FIG. 2 is a functional block diagram of an apparatus for the conversion of algae into various biofuels in accordance with the method 10 of FIG. 1. Referring to FIGS. 1 and 2, the method 10 begins by providing algal biomass that comprises whole algal biomass, residual algal biomass, or both (step 20). The algal biomass is formed from algae. The term “algae” as used herein refers to any organisms with chlorophyll and a thallus not differentiated into roots, steams and leaves, and encompasses prokaryotic organisms such as CYANOBACTERIA (Blue-green algae) and eukaryotic organisms that are photoautotrophic or photoauxotropic. The term “algae” includes macroalgae (commonly known as seaweed) and microalgae. The algal biomass may also comprise dried algae. Algae may include any species or strain in the following taxonomic groups: BACILLARIOPHYTA (diatoms), CHAROPHYTA (stoneworts), CHLOROPHYTA (green algae), CHRYSOPHYTA (golden algae), DINOPHYTA (dinoflagellates), HAEOPHYTA (brown algae), and RHODOPHYTA (red algae).

Algal biomass may be provided by harvesting algae from a source 15 (step 30). The source 15 may be a bioreactor, aquaculture pond, waste water, lake, pond, river, sea, etc. The algae may be, for example, a naturally occurring species, a genetically selected strain, a genetically manipulated strain, a transgenic strain, a synthetic algae, or combinations thereof. Algae grow as a dilute suspension.

The harvested algae are then dewatered (step 40), for example in a concentrator 35 which has an input 25 coupled to the source 15. Concentrator 35 concentrates the algae producing the whole algal biomass at output 45. The whole algal biomass comprises a concentrated algal paste of about 4% to about 12% solids by weight. The term “about”, as used hereinafter, unless otherwise indicated, refers to a value that is no more than 20% above or below the value being modified by the term. The concentrated algal paste is in the form of slurry with a paste-like consistency, able to be pumped from the output 45 of concentrator 35 into a reactor 125 as hereinafter described and shown in FIG. 2. Any one or more known methods for dewatering the algae in the concentrator 35 can be used, including but not limited to, sedimentation, filtration, centrifugation, flocculation, froth floatation, and/or semipermeable membranes. The whole algal biomass is also commercially available from sources such as algal cultivators, Solix Biofuels Inc., Fort Collins, Colo. (USA) and Cyanotech Corp., Hawaii (USA).

To form the residual algal biomass, the whole algal biomass from concentrator 35 (output 45) is dried in a dryer 55 (step 50) by evaporation or the like to provide dried concentrated algal paste at output 65 and also applied to an input of extractor 75. The neutral triacelycerols (TAGs) are then extracted from the dried concentrated algal paste in a neutral lipid extractor 75 (step 60) by known lipid extraction methods using an organic solvent such as hexane or the like to produce a “neutral oil extract” (also referred to herein as “TAG oil”) at output 85. The TAG oil produced at output 85 during the extracting step (step 60) may be withdrawn from the neutral lipid extractor 75 and conventionally processed to produce FAME biodiesel as noted above. A mixture of the organic solvent and residual dried concentrated algal paste is provided at output 95 and to an input of evaporator 105. The organic solvent is evaporated in evaporator 105 (step 70) leaving the residual algal biomass at output 115 which is provided to reactor 125. Steps 50, 60, and 70 are shown in dotted lines in FIG. 1, representing the additional steps needed to provide residual algal biomass to reactor 125.

“Residual algal biomass” refers to the dried bagasse remaining after the neutral lipids, e.g., triacelycerols (TAGs) are recovered as “TAG oil” by the solvent extraction. The residual algal biomass comprises the polar lipids and the glycolipids, residual protein and carbohydrates, and algal cell debris. The total mass of the bagasse after the solvent extraction of TAG oil is at least 70% of the total algal biomass.

Still referring to FIGS. 1 and 2, the algal biomass comprised of whole algal biomass from concentrator 35, the residual algal biomass from evaporator 105, or both, is introduced as feedstock to reactor 125 to be hydrogenolysed (step 80) under reaction conditions sufficient to produce partially deoxygenated lipid-based oil in an organic phase, as hereinafter described. The algal biomass is hydrogenolysed by liquefying the algal biomass in water from a water source 133, in a neutral solvent from a solvent source 135, or both, provided to reactor 125. The amount of water added to the algal biomass comprises about 50 ml to about 100 ml per 100 g of algal biomass. The amount of neutral solvent that may be used comprises about 50 ml to about 100 ml neutral solvent per 100 g of algal biomass. As used herein, a “neutral solvent” means that the neutral solvent does not react with the hydrogen, i.e., that it does not consume the hydrogen used in the method according to exemplary embodiments of the present invention. Exemplary neutral solvents include hexadecane, hexane, cyclohexane, other paraffinic solvents, or a combination thereof. Aromatic solvents such as toluene can also be used as the neutral solvent as the carbon-carbon double bonds therein are generally not hydrogenated under the processing conditions according to exemplary embodiments of the present invention. The neutral solvent promotes the hydrogenolysis reaction as hydrogenolysed algal biomass in the neutral solvent reacts more effectively than hydrogenolysed algal biomass in water. The neutral solvent may be added (step 90) to the reactor during the hydrogenolysing step, slurried with the algal biomass prior to addition to the reactor, or both.

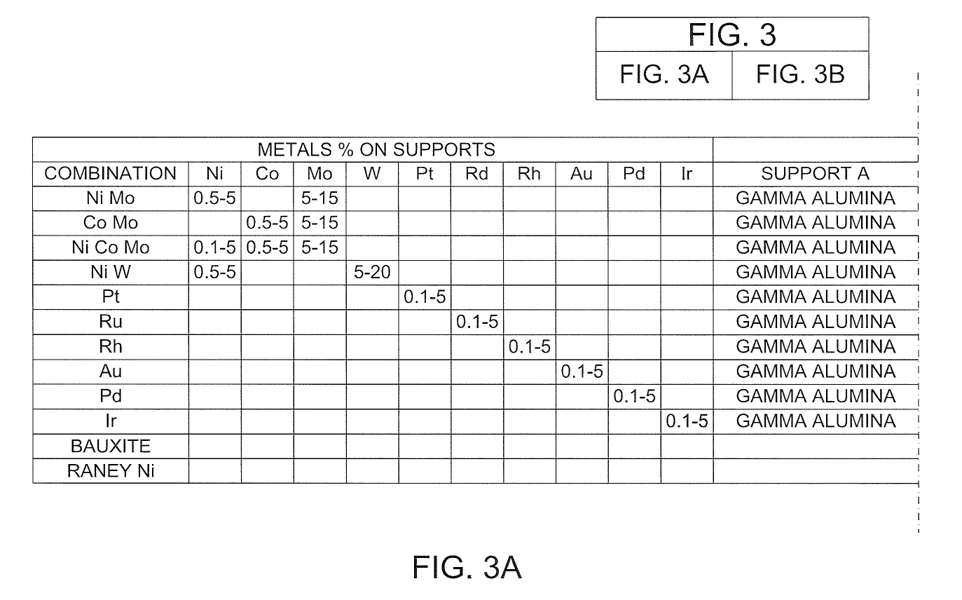

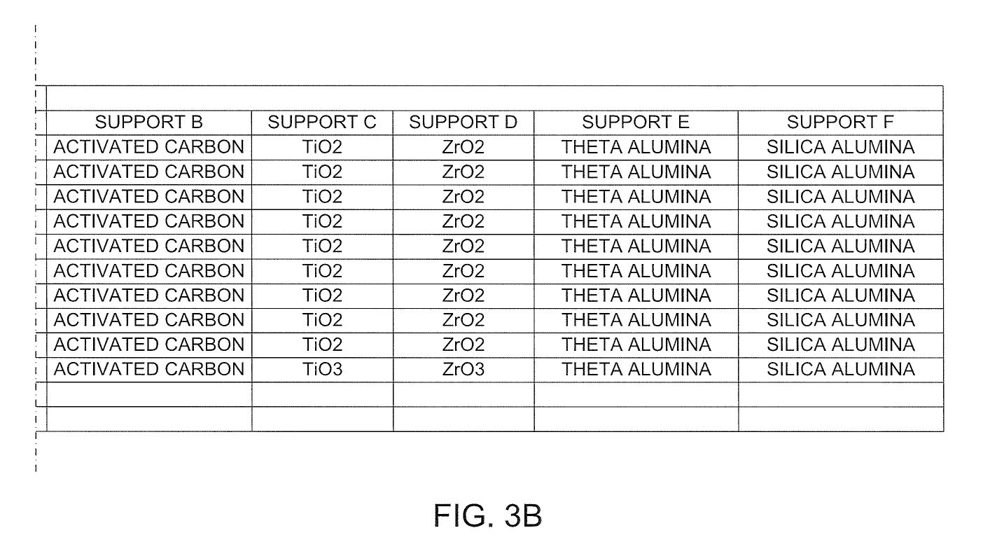

The algal biomass is hydrogenolysed in the presence of a hydrogenolytic catalyst in a hydrogen gas atmosphere at selected reaction conditions as hereinafter described. Suitable exemplary hydrogenolytic catalysts include those shown in the table of FIG. 3 (Supports A-F representative alternative supports).

The quantity of the hydrogenolytic catalyst comprises from about 0.5 g catalyst to about 2.5 g catalyst, preferably about 1 g catalyst to about 2 g catalyst, and most preferably about 1.5 g catalyst, per 100 g of the feedstock. The reaction conditions include an elevated pressure, an elevated pressure, and a selected reaction time. The elevated pressure may range from about 689475.728 Pascal (about 100 psig) to about 2068427.184 Pascal (about 300 psig), preferably from about 1034213.592 Pascal (150 psig) to about 1723689.32 Pascal (250 psig), and most preferably is about 1378951.456 Pascal (200 psig). The elevated temperature ranges from about 200° C. to about 400° C., preferably about 280° C. to about 360° C., and most preferably about 320° C. Reaction time is from about 60 minutes to about 200 minutes, preferably about 80 minutes to about 160 minutes, and most preferably about 120 minutes. The reactor may be a continuously stirred tank reactor (CSTR) or the like.

The hydrogenolysing step occurring in reactor 125 produces an aqueous phase, a solid phase, and as noted above, the organic phase including the partially deoxygenated lipid-based oil. The neutral solvent, if used, may also be in the organic phase. The partially deoxygenated lipid-based oil comprises paraffinic hydrocarbons (the portion deoxygenated with the hydrogen gas forming water) and fatty acids. Fatty acids are oxygenated hydrocarbons derived from the hydrogenolytic splitting of triglycerides. Paraffinic hydrocarbons are straight chain alkanes, consisting of only the elements carbon (C) and hydrogen (H). The aqueous phase contains process residues such as aromatics and nitrogen-containing compounds. The solid phase comprises algal cell debris.

As noted previously, algae generally contain three types of lipids, the stored neutral lipids, primarily triacelycerols (TAGS), the glycolipids found in chloroplast membranes, and the polar lipids, primarily phospholipids, of the plasma membranes. Most phospholipids contain a diglyceride, a phosphate group, and a simple organic molecule such as choline. Catalytic hydrogenolysis (liquefaction) as described above breaks the bond between carbon and the phosphate group to remove the phosphate group from the phospholipid. Glycolipids are lipids with a carbohydrate attached. Catalytic hydrogenolysis (liquefaction) as described above breaks the bond between the carbon and the carbohydrate from the glycolipids to convert the glycolipids into the paraffinic hydrocarbons in the organic phase. Algal biomass also contains cell debris, chlorophylls, carbohydrates and proteins.

After the hydrogenolysing step (step 80), the aqueous phases and solid phases may be phase separated (step 100) from the organic phase comprising the partially deoxygenated lipid-based oil. The aqueous phase may be removed from the reactor at output 155, for example, by decanting the aqueous phase in, for example, an aqueous separator 175. If neutral solvent has been used, the aqueous phase is removed to improve deoxygenation of the fatty acids in the organic phase as hereinafter described. If not removed, water may prevent interaction between the hydroprocessing catalyst and the partially deoxygenated lipid-based oil. If no neutral solvent is used, removal of the aqueous phase from the organic phase is preferred, but optional. The solid phase may be removed from the reactor at output 165, for example, by a solids separator 185 such as a mechanical separator and sent for further processing or removal.

The partially deoxygenated lipid-based oil in the organic phase is then substantially deoxygenated (step 110) to produce hydrocarbon oil at output 195 of reactor 125. The hydrocarbon oil is principally comprised of paraffinic hydrocarbons. As used herein, the phrase “substantially deoxygenated” means that the hydrocarbon oil is between about 80% to about 100% deoxygenated. In one embodiment, the fatty acids in the partially deoxygenated lipid-based oil are substantially deoxygenated into paraffinic hydrocarbons by a hydrogenation reaction in the presence of a hydroprocessing catalyst at an elevated deoxygenation pressure. The hydrocarbon oil produced in accordance with exemplary embodiments thus comprises the paraffinic hydrocarbons produced during the hydrogenolysing step and additional paraffinic hydrocarbons produced during the deoxygenation step. Suitable exemplary hydroprocessing catalysts include a nano Nickel Hydrated Catalytic Technology (HCT)™ catalyst, or other known hydroprocessing catalysts. The hydroprocessing catalyst and the hydrogenolytic catalyst may be the same, including those shown in the table of FIG. 3. In a preferred embodiment, the hydrogenolytic catalyst has a noble metal on the support and the hydroprocessing catalyst has a non-noble metal on the support.

The hydroprocessing catalyst quantity ranges from about 0.1 g to about 5 g per 100 ml of partially deoxygenated lipid-based oil. The partially deoxygenated lipid-based oil is subjected to an elevated deoxygenation pressure from about 6205281.552 Pascal (about 900 psig) to about 8963184.464 Pascal (about 1300 psig), an elevated temperature from about 280° C. to about 350° C., and a reaction time from about 10 hours to about 50 hours, preferably about 20 hours to about 40 hours, and most preferably about 30 hours. In a preferred embodiment, the hydrogenolysing step (step 80) and deoxygenating step (step 110) are subsequent steps performed in the same reactor. In an alternative embodiment, deoxygenating (step 110) may alternatively or additionally be performed in a hydroprocessing reactor (not shown).

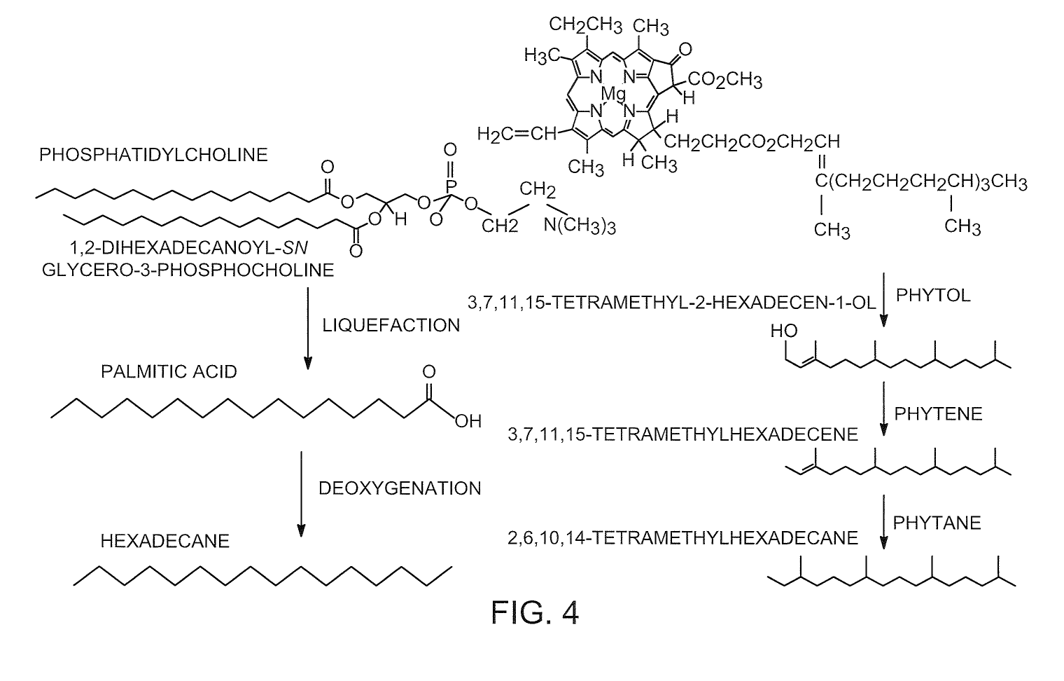

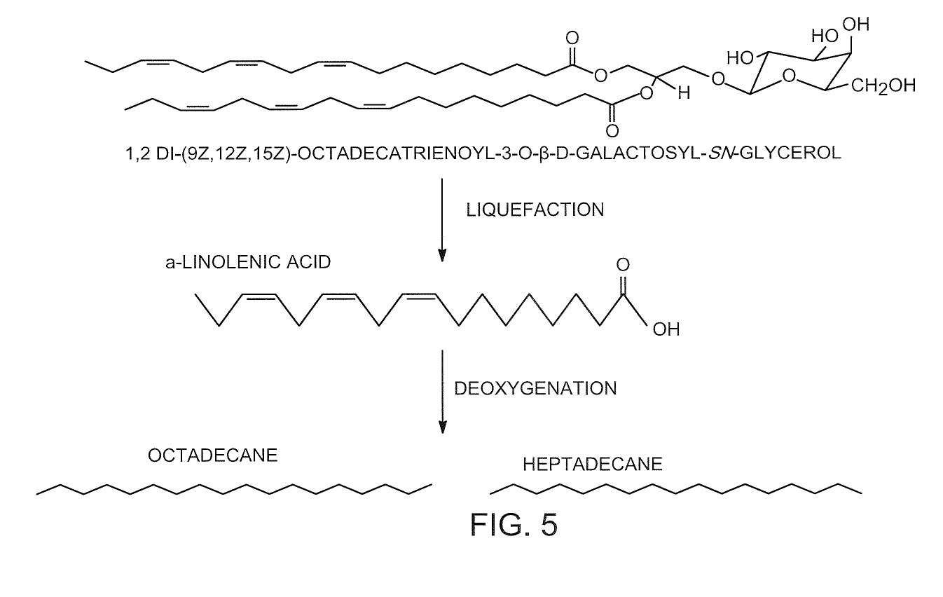

As noted previously, paraffinic hydrocarbons are the principal chemical compounds in the hydrocarbon oil produced in the reactor from hydrogenolysis (e.g., liquefaction) and deoxygenation of algal biomass in accordance with exemplary embodiments of the present invention. FIG. 4 illustrates the exemplary production of the paraffinic hydrocarbons (hexadecane and phytane) from an exemplary phospholipid (phosphatidylcholine) (a polar lipid) contained in the plasma membrane of algal cells and from chlorophyll as a result of hydrogenolysing (liquefying) and deoxygenating residual algal biomass in accordance with exemplary embodiments as just described. FIG. 5 illustrates the exemplary production of the paraffinic hydrocarbons heptadecane and octadecane from an exemplary glycolipid (monogalactosyldiacylglycerol) contained in the chloroplast membranes of algae as a result of hydrogenolysing (liquefying) and deoxygenating the residual algal biomass.

While the production of the paraffinic hydrocarbons hexadecanes, octadecanes, phytanes, and heptadecanes are described, the present invention is not so limited. Other paraffinic hydrocarbons including normal paraffins having between eight and fourteen carbons may also be obtained by hydrogenolysing and deoxygenating algal biomass. In addition, other valuable chemical compounds are also produced in the hydrocarbon oil. Hydrocarbon oil produced in accordance with exemplary embodiments described herein can be used directly as biofuel, and/or as a feedstock for further processing into a transportation biofuel such as biodiesel, biojet fuel, and biogasoline. That is, hydrocarbon oil from reactor 125 (output 195) may be provided to a gasoline refinery 215 to produce biogasoline at output 225, to a diesel refinery 245 to produce biodiesel at output 255, and/or to a jet fuel refinery 275 to produce biojet fuel at output 285. For example, the hydrocarbon oil may be converted by traditional hydrocracking, isomerization, fractionation, or the like into biogasoline. While the production of hydrocarbon oil as biofuel and as a feedstock for the production of biofuel has been described, the invention is not so limited. The hydrocarbon oil produced in accordance with exemplary embodiments as described herein may be used as a feedstock to produce chemicals as well as biofuels.

### Examples

The following are examples of the production of hydrocarbon oil containing paraffinic hydrocarbons from algal biomass, in accordance with exemplary embodiments described herein. The examples are provided for illustration purposes only, and are not meant to limit the various embodiments of the present invention in any way.

A partially deoxygenated lipid-based oil was produced by liquefaction of residual algal biomass in water using the following liquefaction conditions:

100 g of Dried Algal Biomass (i.e., Residual algal biomass”) in 60 ml Water

1378951.456 Pascal (200 psig) H₂

320° C.

1% Pd/carbon

Catalyst to feedstock ratio: 1.5 g/100 g feed

Reaction time: 120 minutes

˜40% yield of oil.

The partially deoxygenated lipid-based oil was further deoxygenated under the following deoxygenation conditions:
280° C.-320° C.
HCT catalyst
5 g-10 g catalyst/130 ml oil
1-5 hours reaction time
6205281.552 Pascal-8963184.464 Pascal (900-1300 psig) hydrogen

Accordingly, methods for producing biofuels from algal biomass have been provided. From the foregoing, it is to be appreciated that the exemplary embodiments of the method for producing biofuels from algae produce a hydrocarbon product that is substantially deoxygenated making it useful for processing into biofuel. Such deoxygenation occurs without the use of caustic chemicals. More of the lipid material available in the algae is converted to a hydrocarbon product that can be used and/or processed into biofuel thus increasing the useful oil yield as compared to conventional lipid extraction methods.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for producing a hydrocarbon oil from algal biomass comprising the steps of:
hydrogenolysing the algal biomass under reaction conditions sufficient to produce a partially deoxygenated lipid-based oil; and
substantially deoxygenating the partially deoxygenated lipid-based oil.

2. The method of claim 1, wherein the step of hydrogenolysing comprises hydrogenolysing in the presence of a hydrogenolytic catalyst in a quantity comprising from about 0.5 g catalyst to about 2.5 g catalyst per 100 g of algal biomass.

3. The method of claim 1, wherein the step of hydrogenolysing comprises hydrogenolysing with hydrogen gas under the following reaction conditions:
an elevated pressure comprising from about 689475.728 Pascal (about 100 psig) to about 2068427.184 Pascal (about 300 psig);
an elevated temperature from about 200° C. to about 400° C.; and
a reaction time from about 60 minutes to about 200 minutes.

4. The method of claim 1, wherein the step of hydrogenolysing comprises hydrogenlysing whole algal biomass, residual algal biomass, or both.

5. The method of claim 4, further comprising the step of extracting neutral lipids from the algal biomass prior to the hydrogenolysing step to produce a neutral oil extract and the residual algal biomass.

6. The method of claim 1, wherein the step of hydrogenolysing produces an organic phase comprising the partially deoxygenated lipid-based oil, an aqueous phase, and a solid phase.

7. The method of claim 6, further comprising the step of separating the organic phase from the aqueous and solid phases prior to the deoxygenating step.

8. The method of claim 1, wherein the step of hydrogenolysing comprises hydrogenolysing in a neutral solvent producing the partially deoxygenated lipid-based oil in the neutral solvent.

9. The method of claim 8, wherein the step of hydrogenolysing comprises hydrogenolysing in the neutral solvent selected from the group consisting of hexadecane, water, hexane, and a combination thereof.

10. The method of claim 8, further comprising the step of separating the partially deoxygenated lipid-based oil in the neutral solvent from the aqueous and solid phases.

11. The method of claim 1, wherein the step of hydrogenolysing and the step of deoxygenating are performed in the same reactor, in different reactors, or both.

12. A method for producing a hydrocarbon oil from algal biomass, the method comprising the steps of:
liquefying the algal biomass in the presence of a hydrogenolytic catalyst and hydrogen gas to produce a partially deoxygenated lipid-based oil; and
substantially deoxygenating the partially deoxygenated lipid-based oil using a hydroprocessing catalyst to produce the hydrocarbon oil.

13. The method of claim 12, wherein the step of liquefying comprises liquefying to convert neutral lipids, polar lipids, and glycolipids from whole algal biomass, to convert polar lipids and glycolipids from residual algal biomass, or both, into paraffins in the hydrocarbon oil.

14. The method of claim 12, wherein the step of liquefying comprises liquefying in the presence of the hydrogenolytic catalyst in a quantity comprising from about 0.5 g catalyst to about 2.5 g catalyst per 100 g of algal biomass.

15. The method of claim 14, wherein the step of liquefying comprises liquefying at an elevated pressure comprising from about 689475.728 Pascal (about 100 psig) to about 2068427.184 Pascal (about 300 psig), an elevated temperature from about 200° C. to about 400° C., and a reaction time from about 60 minutes to about 200 minutes.

16. The method of claim 12, wherein the step of liquefying produces an organic phase comprising the partially deoxygenated lipid-based oil, an aqueous phase, and a solid phase, the method further comprising the step of separating the organic phase from the aqueous and solid phases prior to the deoxygenating step.

17. The method of claim 12, wherein the step of liquefying comprises liquefying in a neutral solvent producing the partially deoxygenated lipid-based oil in the neutral solvent.

18. The method of claim 17, wherein the step of liquefying comprises liquefying in the neutral solvent selected from the group consisting of hexadecane, water, hexane, and a combination thereof.

19. The method of claim 17, further comprising the step of separating the partially deoxygenated lipid-based oil in the neutral solvent from the aqueous and solid phases.

20. A method for producing liquid hydrocarbons from algal biomass, the method comprising the steps of:
liquefying whole algal biomass, residual algal biomass, or both, in the presence of a hydrogenolytic catalyst in a hydrogen atmosphere at an elevated temperature and pressure to produce an organic phase comprising a partially deoxygenated lipid-based oil, an aqueous phase, and a solid phase;
removing the aqueous and solid phases from the partially deoxygenated lipid-based oil; and
substantially deoxygenating the partially deoxygenated lipid-based oil using a hydroprocessing catalyst.
